(19) United States
(12) Patent Application Publication
(10) Pub. No.: US 2014/0336380 A1
(43) Pub. Date: Nov. 13, 2014

(54) PROCESS FOR THE PREPARATION OF AGOMELATINE

(71) Applicant: Ranbaxy Laboratories Limited, New Delhi, Delhi (IN)

(72) Inventors: Anu Mittal, Kurukshetra (IN); Mahavir Singh Khanna, New Delhi (IN); Rajesh Kumar Thaper, Jammu (IN); Mohan Prasad, Gurgaon (IN); Sudershan Kumar Arora, Gurgaon (IN)

(21) Appl. No.: 14/360,723

(22) PCT Filed: Nov. 21, 2012
(86) PCT No.: PCT/IB2012/056600

§ 371 Date: May 27, 2014

(30) Foreign Application Priority Data
Dec. 1, 2011 (IN) 3464/DEL/2011

(51) Int. Cl. C07C 213/02; C07D 487/18

(52) U.S. Cl. CPC C07C 213/02; C07D 487/18
USPC 544/215; 564/378

(57) ABSTRACT

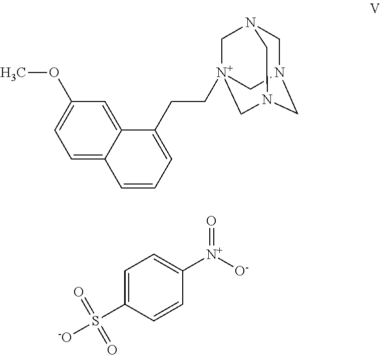

The present invention provides a process for the preparation of agomelatine and its intermediate compounds. The invention also provides an intermediate compound of agomelatine represented by Formula (V).

### FIELD OF THE INVENTION

The present invention provides a process for the preparation of agomelatine, represented by Formula I and its intermediate compounds. The invention also provides an intermediate compound of agomelatine, represented by Formula V.

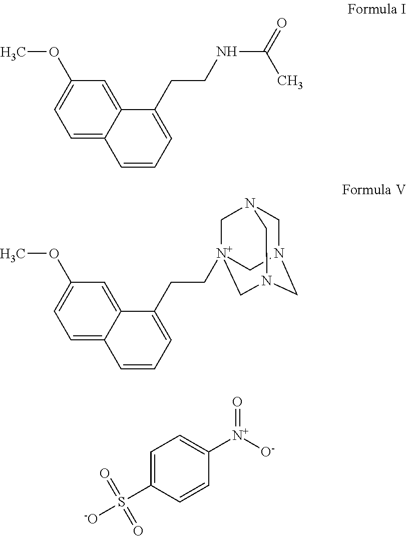

### BACKGROUND OF THE INVENTION

Agomelatine is chemically known as N-[2-(7-methoxynaphthalen-1-yl)ethyl]acetamide. It is indicated for the treatment of major depressive episodes in adults.

Agomelatine and its preparation are disclosed in U.S. Pat. No. 5,225,442. The preparation comprises converting (7-methoxy-1-naphthyl)acetic acid to (7-methoxy-1-naphthyl)ethanamine via the preparation of (7-methoxy-1-naphthyl)acetamide or (7-methoxy-1-naphthyl)acetonitrile intermediate compounds.

U.S. Pat. Nos. 7,476,751; 7,479,569; and 7,470,806 and U.S. Publication Nos. 2010/0137628 and 2010/0036161 describe a process for the preparation of agomelatine comprising a reduction of (7-methoxy-1-naphthyl)acetonitrile to (7-methoxy-1-naphthyl)ethylamine, followed by an acetylation step.

U.S. Publication No. 2011/0130571 describes a process for the preparation of agomelatine comprised of reacting 7-methoxy-1-naphthyl ethanol with benzene sulfonyl chloride to obtain 7-methoxy-1-naphthylethyl benzene sulfonate and condensing it with potassium phthalimide, followed by sequential hydrolysis and acetylation steps.

The present invention provides an alternate process for the preparation of agomelatine and its intermediate compounds.

### SUMMARY OF THE INVENTION

The present invention provides a process for the preparation of agomelatine comprising the use of 1-[2-(7-methoxynaphthalen-1-yl)ethyl]-3,5,7-triaza-1-azoniatricyclo[3.3.1.1^{3,7}]decane 4-nitrobenzene sulfonate (Formula V) as an intermediate compound.

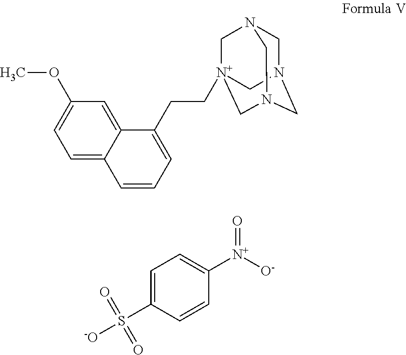

The present invention also provides the compound of Formula V.

### DETAILED DESCRIPTION OF THE INVENTION

The term “organic solvent”, as used herein, includes dichloromethane, ethyl acetate, butyl acetate, dichloroethane, tetrahydrofuran, acetonitrile, acetone, cyclohexane, toluene, chloroform, 1,4-dioxane, dimethylsulfoxide, dimethylformamide, methanol, ethanol, propanol, butanol, and the like.

The term “base”, as used herein, is meant to include organic bases (for example pyridine, triethylamine, and the like) and/or inorganic bases (for example, sodium hydride, ammonium hydroxide, sodium carbonate, and the like). Some non-limiting examples of “base” are sodium hydroxide, potassium hydroxide, magnesium hydroxide, dipotassium hydrogen orthophosphate, magnesium carbonate, sodium carbonate, potassium carbonate, pyridine, trimethylamine, triethylamine, diisopropylethylamine, and/or N-methyl morpholine.

The term “about”, as used herein, when used along with values assigned to certain measurements and parameters means a variation of 10% from such values, or in the case of a range of values, means a 10% variation from both the lower and upper limits of such ranges.

The present invention can be explained by way of the following aspects.

A first aspect of the present invention provides a process for the preparation of agomelatine comprising a step of converting 1-[2-(7-methoxynaphthalen-1-yl)ethyl]-3,5,7-triaza-1-azoniatricyclo[3.3.1.1^{3,7}]decane 4-nitrobenzene sulfonate (Formula V)

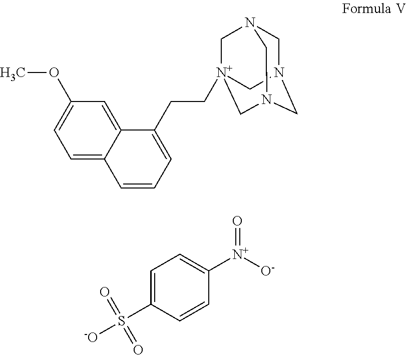

to 2-(7-methoxynaphthalen-1-yl)ethanamine (Formula VI) or its salt.

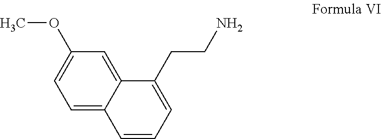

In an embodiment of this aspect, 2-(7-methoxynaphthalen-1-yl)ethanamine (Formula VI) or its salt is acetylated to obtain agomelatine.

In another embodiment, the compound of Formula VI or its salt can be acetylated using acetyl chloride or acetic anhydride.

In another embodiment, 1-[2-(7-methoxynaphthalen-1 -yl)ethyl]-3,5,7-triaza-1-azoniatricyclo[3.3.1.1^{3,7}]decane 4-nitrobenzene sulfonate (Formula V) is treated with an acid to obtain the compound of Formula VI or its salt.

In another embodiment, the acid can be selected from the group comprised of sulfuric acid, phosphoric acid, methanesulfonic acid, and hydrochloric acid.

Accordingly, 1-[2-(7-methoxynaphthalen-1-yl)ethyl]-3,5,7-triaza-1-azoniatricyclo [3.3.1.1^{3,7}]decane 4-nitrobenzene sulfonate (Formula V) is treated with concentrated hydrochloric acid in the presence of methanol to obtain 2-(7-methoxynaphthalen-1-yl)ethanamine hydrochloride (Formula VIa).

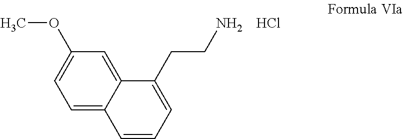

The hydrochloride salt is then acetylated to provide agomelatine.

A second aspect of the present invention provides a process for the preparation of agomelatine comprising a step of converting 1-[2-(7-methoxynaphthalen-1-yl)ethyl]-3,5,7-triaza-1-azoniatricyclo [3.3.1.1^{3,7}] decane 4-nitrobenzene sulfonate (Formula V)

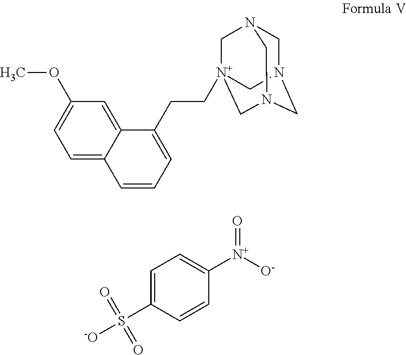

into a carbon dioxide adduct of 2-(7-methoxynaphthalen-1-yl)ethanamine (Formula VII).

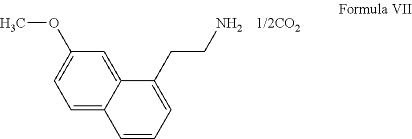

In an embodiment of this aspect, the carbon dioxide adduct of Formula VII is converted into agomelatine.

In another embodiment, the conversion of the compound of Formula V into the compound of Formula VII can be performed with or without isolating the 2-(7-methoxynaphthalen-1-yl)ethanamine intermediate compound of Formula VI or its salt.

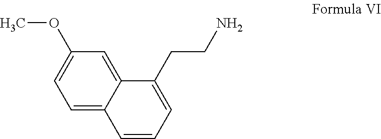

In another embodiment, 1-[2-(7-methoxynaphthalen-1-yl)ethyl]-3,5,7-triaza-1-azoniatricyclo[3.3.1.1^{3,7}]decane 4-nitrobenzene sulfonate (Formula V) is treated with an acid, with or without isolating the intermediate compound of Formula VI or its salt, and then treated with carbon dioxide to provide the carbon dioxide adduct of 2-(7-methoxynaphthalen-1-yl)ethanamine (Formula VII).

In another embodiment, the acid can be selected from the group comprised of sulfuric acid, phosphoric acid, methanesulfonic acid, and hydrochloric acid.

The compound of Formula VI or its salt can be dissolved in an organic solvent and treated with carbon dioxide at a temperature range of about 0° C. to about 30° C. to obtain the adduct represented by Formula VII. This adduct is acetylated to obtain agomelatine.

The acetylation of the adduct can be carried out using an acetylating agent (for example, acetyl chloride or acetic anhydride) in the presence of an organic solvent.

Accordingly, 1-[2-(7-methoxynaphthalen-1-yl)ethyl]-3,5,7-triaza-1-azoniatricyclo[3.3.1.1^{3,7}]decane 4-nitrobenzene sulfonate (Formula V) is treated with concentrated hydrochloric acid in the presence of methanol to obtain 2-(7-methoxynaphthalen-1-yl)ethanamine hydrochloride (Formula VIa). The hydrochloride salt is basified and then treated with carbon dioxide to provide the carbon dioxide adduct of 2-(7-methoxynaphthalen-1-yl)ethanamine (Formula VII). The compound of Formula VII is acetylated using acetic anhydride in the presence of methanol to obtain agomelatine.

A third aspect of the present invention provides a process for the preparation of agomelatine comprising the step of converting 2-(7-methoxynaphthalen-1-yl)ethyl 4-nitrobenzene sulfonate of Formula IV

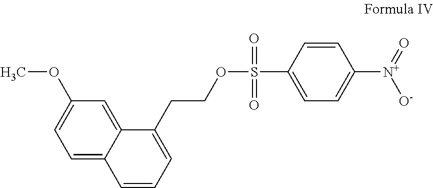 [SMILES: COc1ccc2cccc(CCOS(=O)(=O)c3ccc([N+](=O)[O-])cc3)c2c1]

into 1-[2-(7-methoxynaphthalen-1-yl)ethyl]-3,5,7-triaza-1-azoniatricyclo[3.3.1.1^{3,7}]decane 4-nitrobenzene sulfonate of Formula V.

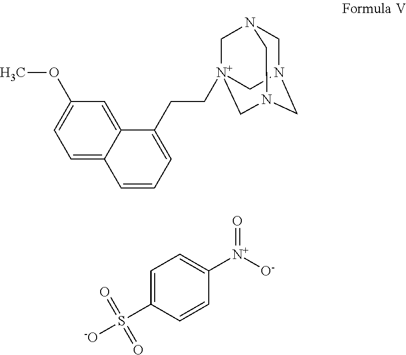

In one embodiment, the compound of Formula IV is treated with hexamethylene tetramine to obtain the compound of Formula V.

The 1-[2-(7-methoxynaphthalen-1-yl)ethyl]-3,5,7-triaza-1-azoniatricyclo[3.3.1.1^{3,7}]decane 4-nitrobenzene sulfonate compound of Formula V can be converted into agomelatine by following the process described hereinabove in the first or second aspect of the present invention.

Accordingly, 2-(7-methoxynaphthalen-1-yl)ethyl 4-nitrobenzene sulfonate (Formula IV) is condensed with hexamethylene tetramine and dichloromethane to obtain 1-[2-(7-methoxynaphthalen-1-yl)ethyl]-3,5,7-triaza-1-azoniatricyclo[3.3.1.1^{3,7}]decane 4-nitrobenzene sulfonate (Formula V). The compound of Formula V is then treated with concentrated hydrochloric acid in the presence of methanol to obtain 2-(7-methoxynaphthalen-1 -yl)ethanamine hydrochloride (Formula VIa). The hydrochloride salt is then acetylated to provide agomelatine. Alternatively, the hydrochloride salt of Formula VIa is basified and then treated with carbon dioxide to provide a carbon dioxide adduct of 2-(7-methoxynaphthalen-1-yl)ethanamine (Formula VII). The compound of Formula VII is acetylated using acetic anhydride in the presence of methanol to obtain agomelatine.

A fourth aspect of the present invention provides 1-[2-(7-methoxynaphthalen-1-yl)ethyl]-3,5,7-triaza-1-azoniatricyclo[3.3.1.1^{3,7}] decane 4-nitrobenzene sulfonate of Formula V.

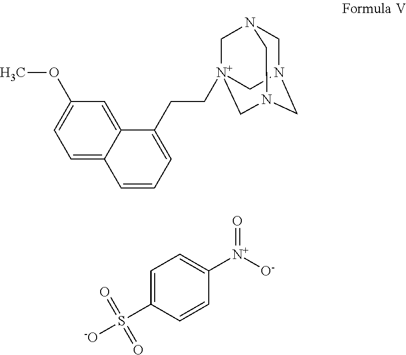

In one embodiment, the compound of Formula V can be used as an intermediate for the preparation of agomelatine.

The agomelatine can be prepared by making use of the compound of Formula V as described hereinabove in the first and second aspects of the present invention.

The starting material, 2-(7-methoxynaphthalen-1-yl)ethyl 4-nitrobenzene sulfonate of Formula IV, can be prepared by treating (7-methoxy-1-naphthyl)ethanol of Formula III

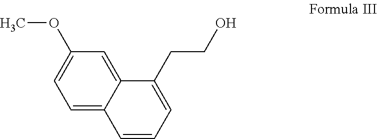

with 4-nitrobenzene sulfonyl chloride in the presence of a base and an organic solvent. The (7-methoxy-1-naphthyl)ethanol can be obtained by treating 7-methoxy-1-naphthyl acetic acid, represented by Formula II

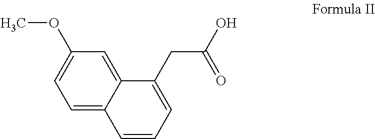 [SMILES: COc1ccc2cccc(CC(=O)O)c2c1]

with lithium aluminum hydride in the presence of tetrahydrofuran. The 7-methoxy-1-naphthyl acetic acid of Formula II can be prepared by any method known in the art, for example, by following the process described in U.S. Pat. No. 5,225,442.

While the present invention has been described in terms of its specific aspects, certain modifications and equivalents will be apparent to those skilled in the art and are intended to be within the scope of the present invention.

In the following section, aspects are described by way of examples to illustrate the processes of the invention. However, these are not intended in any way to limit the scope of the present invention. Variants of these examples would be evident to persons ordinarily skilled in the art.

### EXAMPLES

### Example 1

### Preparation of (7-Methoxy-1-Naphthyl)Ethanol

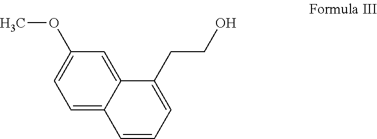

To a solution of 7-methoxy-1-naphthyl acetic acid (Formula II, 60 g) in tetrahydrofuran (240 mL), a suspension of lithium aluminum hydride (16.8 g) in tetrahydrofuran (1500 mL) was slowly added over a period of 15 to 20 minutes at 0° C. The reaction mixture was stirred for 4 hours at 25° C. to 30° C. After completion of the reaction, ethyl acetate (120 mL) was added, followed by the slow addition of 5N hydrochloric acid (100 mL) over a period of 30 minutes at 0° C. The reaction mixture was stirred and the layers were allowed to settle. The organic layer was separated and removed under vacuum (40° C. to 45° C., ≠100 mbar). The residue so obtained was used for the next example.

### Example 2

### Preparation of 2-(7-Methoxynaphthalen-1-Yl)Ethyl 4-Nitrobenzene Sulfonate

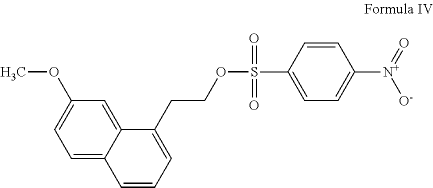 [SMILES: COc1ccc2cccc(CCOS(=O)(=O)c3ccc([N+](=O)[O-])cc3)c2c1]

To a mixture of (7-methoxy-1-naphthyl)ethanol (Formula III, obtained in Example 1), triethyl amine (55 g), and dichloromethane (550 mL); 4-nitrobenzene sulfonyl chloride (72.3 g) was added in small lots at 0° C. The reaction mixture was stirred and heated to 25° C. to 30° C. After completion of the reaction, 2.5% sodium hydroxide solution (200 mL) was added at 25° C. to 30° C. The reaction mixture was stirred and the product was extracted with the addition of dichloromethane (550 mL). The organic layer was concentrated under vacuum. The residue so obtained was stirred in toluene (165 mL) and then in methanol (165 mL) for 30 minutes. This was filtered and the product obtained was dried under vacuum (5-10 mbar) at 45° C. to 50° C.

Yield (w/w): 83%

### Example 3

### Preparation of 1-[2-(7-Methoxynaphthalen-1-Yl)Ethyl]-3,5,7-Triaza-1-Azoniatricyclor[3.3.1.1^{3,7}]Decane 4-Nitrobenzene Sulfonate

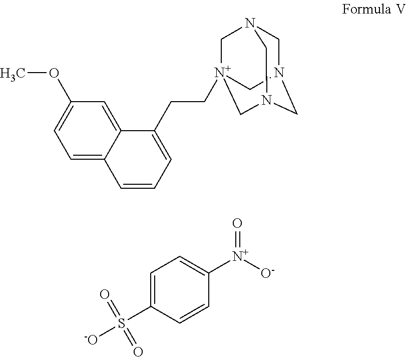

Hexamethylene tetramine (92.3 g) was added to a solution of 2-(7-methoxynaphthalen-1-yl)ethyl-4-nitrobenzene sulfonate (Formula IV, obtained in Example 2, 85 g) in dichloromethane (425 mL). The reaction mixture was refluxed for 18 hours and cooled at 25° C. to 30° C. The solid separated was filtered and further stirred with de-ionized water (255 mL) at pH 4 to 5 (adjusted using 15 mL to 20 mL of concentrated HCl) for one hour. The reaction mixture was filtered and the product obtained was dried under vacuum (5-10 mbar) at 45° C. to 50° C. for 10 to 15 hours.

Yield (w/w): 59.26%

### Example 4

### Preparation of 2-(7-Methoxynaphthalen-1-Yl)Ethanamine Carbon Dioxide Adduct

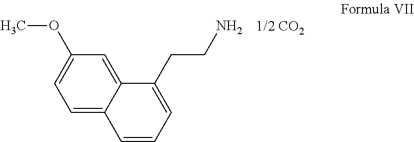

To a solution of 1-[2-(7-methoxynaphthalen-1-yl)ethyl]-3,5,7-triaza-1-azoniatricyclo[3.3.1.1^{3,7}]decane 4-nitrobenzene sulfonate (Formula V, obtained in Example 3, 5 g) in methanol (30 mL) was slowly added concentrated hydrochloric acid (2.5 mL). The reaction mixture was heated up to the reflux temperature and stirred for 10 minutes. After completion of the reaction, the reaction mixture was cooled to 30° C. to 35° C. The salt separated was filtered and the mother liquor was concentrated under vacuum. The residue so obtained was extracted in de-ionized water (20 mL). The aqueous layer was basified using 5% NaOH (2 mL) and extracted sequentially with ethyl acetate (20 mL, pH 5), dichloromethane (20 mL, pH 7) and finally with toluene (50 mL, pH 13). The toluene layer was then purged with excess of carbon dioxide gas for 12 to 15 hours at 25° C. to 30° C. to obtain a solid. The solid was dried under vacuum (5-10 mbar) at 30° C. to 35° C. over 10 to 15 hours to obtain the title product.

Yield (w/w): 33%

### Example 4a

### Preparation of 2-(7-Methoxynaphthalen-1-Yl)Ethanamine Hydrochloride

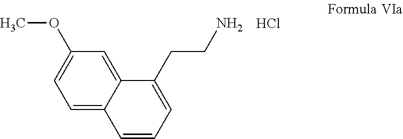

To a solution of 1-[2-(7-methoxynaphthalen-1-yl)ethyl]-3,5,7-triaza-1-azoniatricyclo[3.3.1.1^{3,7}]decane 4-nitrobenzene sulfonate (Formula V, obtained in Example 3, 10 g) in methanol (60 mL) was slowly added concentrated hydrochloric acid (10 mL). The reaction mixture was heated to reflux temperature and stirred for 90 minutes. After completion of the reaction, the reaction mixture was cooled to 30° C. to 35° C. The salt separated and was filtered, and the mother liquor was concentrated under vacuum (200-220 mbar). The residue so obtained was basified initially with 5% NaHCO₃ solution (100 mL) and then with 5% NaOH (5 mL, pH 13) and extracted in dichloromethane (100 mL). The dichloromethane layer was concentrated under a vacuum (400-420 mbar) at 40° C. The residue so obtained was dissolved in ethyl acetate (20 mL), acidified with concentrated HCl (9 mL to 12 mL), and extracted with water (30 mL) at a pH 4.5 to pH 5. The aqueous layer was again basified with 5% NaOH (8 mL to 12 mL, pH 13) and the product was extracted in ethyl acetate. The ethyl acetate layer was again acidified with concentrated HCl (7 mL to 9 mL, pH 2). The product was recovered under vacuum (200-220 mbar) at 45° C. to 50° C. and column chromatographed using 30%

CH₃OH/ethyl acetate, and resulted in a solid product after evaporating. The solid obtained was dried under vacuum (5-10 mbar) at 40° C. to 45° C. over 10 to 15 hours to obtain the title product.

Yield (w/w): 46%

### Example 4b

### Preparation of 2-(7-Methoxynaphthalen-1-Yl)Ethanamine Hydrochloride

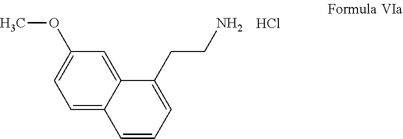

A solution of (7-methoxynaphthalen-1-yl)acetonitrile (30 g) in methanol (150 mL) and aqueous ammonia (15 mL) was treated with hydrogen gas (3 Kg) in the presence of Raney nickel (45 g) at 40° C. After completion of the reaction, the reaction mixture was filtered through a Hyflo®. The filtrate was concentrated under vacuum (200-220 mbar) at 45° C. to 50° C. to obtain a residue. The residue in ethyl acetate (60 mL) was acidified (pH 2) with concentrated hydrochloric acid (10 mL to 15 mL) at 10° C. to 15° C. to obtain a solid. The solid was dried under vacuum (5-10 mbar) at 45° C. to 50° C. over 10 to 15 hours to get the title product.

Yield (w/w): 32%

### Example 5

### Preparation of Agomelatine

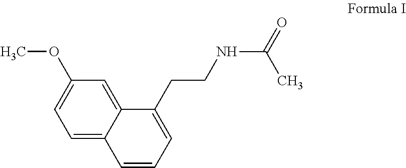 [SMILES: COc1ccc2cccc(CCNC(C)=O)c2c1]

To a mixture of 2-(7-methoxynaphthalen-1-yl)ethanamine hydrochloride (Formula VIa, obtained in Example 4a, 1.5 g) and sodium acetate (0.57 g) in methanol (9 mL); acetic anhydride (0.68 g) was added drop-wise. The reaction mixture was heated to reflux and stirred for 3 to 4 hours. After completion of the reaction, the reaction mixture was cooled to 30° C. to 35° C. The salt separated was filtered and the mother liquor was concentrated under vacuum (200-220 mbar) at 45° C. to 50° C. The residue so obtained was extracted in dichloromethane/water (20/10 mL). The organic layer was concentrated under vacuum (400-420 mbar) at 40° C. and crystallized in diisopropyl ether (15 mL). The solid obtained was dried under vacuum (5-10 mbar) at 45° C. to 50° C. over 10 to 15 hours to obtain the title product.

Yield (w/w): 52%

### Example 6

### Preparation of Agomelatine

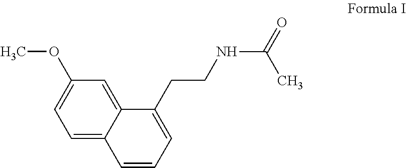 [SMILES: COc1ccc2cccc(CCNC(C)=O)c2c1]

To a mixture of 2-(7-methoxynaphthalen-1 -yl)ethanamine carbon dioxide adduct (Formula VII, obtained in Example 4, 0.6 g) and sodium acetate (0.24 g) in methanol (9 mL); acetic anhydride (0.3 g) was added drop-wise. The reaction mixture was heated to reflux and stirred for 1 to 2 hours. After completion of the reaction, the reaction mixture was cooled to 30° C. to 35° C. Ice (18 g) was added to the reaction mixture and stirred for another 2 hours at 10° C. to 15° C. The solid product was filtered and dried under vacuum (5-10 mbar) at 45° C. to 50° C. over 10 to 15 hours to obtain the title product.

Yield (w/w): 91%

1. A process for the preparation of agomelatine comprising a step of converting 1-[2-(7-methoxynaphthalen-1-yl)ethyl]-3 ,5,7-triaza-1 -azoniatricyclo [3.3.1.1^{3,7]decane} 4-nitrobenzene sulfonate (Formula V) 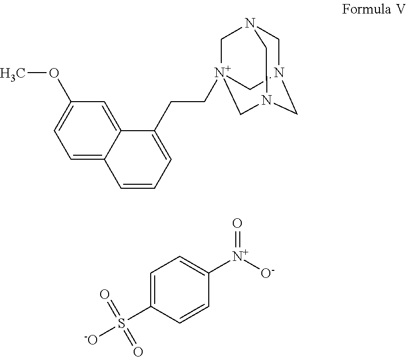
into 2-(7-methoxynaphthalen-1-yl)ethanamine (Formula VI) or its salt.

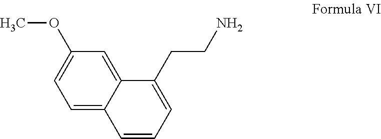

2. The process of claim 1, wherein 2-(7-methoxynaphthalen-1-yl)ethanamine (Formula VI) or its salt is acetylated to obtain agomelatine.

3. The process of claim 2, wherein the compound of Formula VI or its salt is acetylated using acetyl chloride or acetic anhydride.

4. The process of claim 1, wherein the compound of Formula V is treated with an acid to obtain the compound of Formula VI or its salt.

5. The process of claim 4, wherein the acid is selected from the group comprised of sulfuric acid, phosphoric acid, methanesulfonic acid, and hydrochloric acid.

6. A process for the preparation of agomelatine comprising a step of converting 1-[2-(7-methoxynaphthalen-1 -yl)ethyl]-3 ,5,7-triaza-1 -azoniatricyclo [3.3.1.1^{3,7}]decane 4-nitrobenzene sulfonate (Formula V) 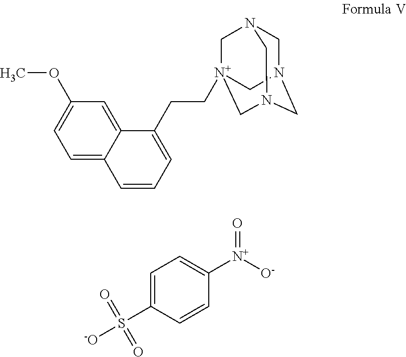
into carbon dioxide adduct of 2-(7-methoxynaphthalen-1-yl)ethanamine (Formula VII).

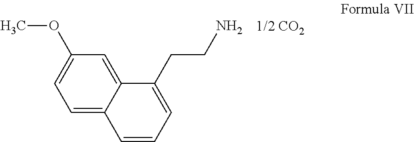

7. The process of claim 6, wherein the carbon dioxide adduct (Formula VII) is further converted into agomelatine.

8. The process of claim 6, wherein the conversion of compound of Formula V into the compound of Formula VII can be performed with or without isolating 2-(7-methoxynaphthalen-1-yl)ethanamine intermediate compound of Formula VI or its salt.

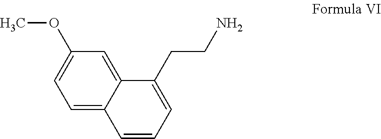

9. The process of claim 6, wherein the compound of Formula V is treated with an acid followed by treatment of carbon dioxide to obtain the compound of Formula VII.

10. The process of claim 9, wherein the acid is selected from the group comprised of sulfuric acid, phosphoric acid, methanesulfonic acid, and hydrochloric acid.

11. A process for the preparation of agomelatine comprising a step of converting 2-(7-methoxynaphthalen-1-yl)ethyl 4-nitrobenzene sulfonate of Formula IV 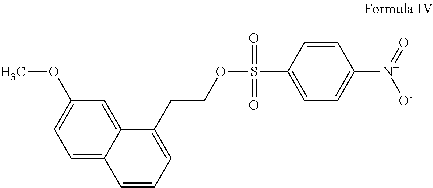 [SMILES: COc1ccc2cccc(CCOS(=O)(=O)c3ccc([N+](=O)[O-])cc3)c2c1]
into 1-[2-(7-methoxynaphthalen-1-yl)ethyl]-3,5,7-triaza-1-azoniatricyclo [3.3.1.1^{3,7}]decane 4-nitrobenzene sulfonate of Formula V.

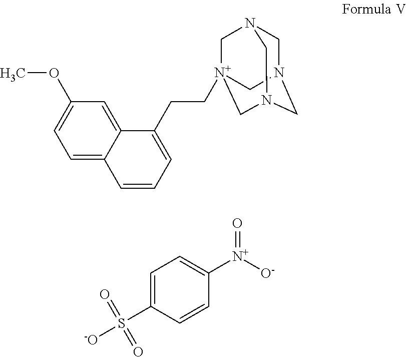

12. The process of claim 11, wherein the compound of Formula IV is treated with hexamethylene tetramine to obtain the compound of Formula V.

13. The process of claim 11, wherein the compound of Formula V is further converted into agomelatine.

14. A compound 1-[2-(7-methoxynaphthalen-1-yl)ethyl]-3,5,7-triaza-1-azoniatricyclo[3.3.1.1^{3,7}]decane 4-nitrobenzene sulfonate of Formula V. [chemical structure]
